(19) United States
(12) Patent Application Publication
(10) Pub. No.: US 2023/0181603 A1
(43) Pub. Date: Jun. 15, 2023

(54) METHODS OF TREATING CANCER WITH METABOLITE ADJUSTMENTS

(71) Applicant: Gus Lawrence, Portland, OR (US)

(72) Inventor: Gus Lawrence, Portland, OR (US)

(21) Appl. No.: 17/550,706

(22) Filed: Dec. 14, 2021

(51) Int. Cl. A61K 31/593; A61K 31/22; A61P 35/00; A61K 31/455; A61K 31/375

(52) U.S. Cl. CPC A61K 31/593; A61K 31/22; A61P 35/00; A61K 31/455; A61K 31/375

(57) ABSTRACT

Cancer treatment methods including adjusting t to in vivo concentration of a selected metabolite in a patient to levels effective to activate selected metabolic pathways that promote differentiation of cancer cells. In some examples the methods for treating cancer include adjusting the in vivo concentration of one or more of vitamin-D, niacin, ascorbic acid, ascorbyl palmitate, and butyrate.

### BACKGROUND

The present disclosure relates generally to methods for treating cancer. In particular, methods of treating cancer with metabolite adjustments to activate selected metabolic pathways are described.

Despite significant advances in cancer care, there is no effective therapy for gliomas and glioblastomas. Glioblastomas are extremely aggressive, and the average survival time is around 12-18 months. Tumor cells are highly motile and migrate long distances from the original tumor site.

Tumor cells fail to differentiate in a way similar to non-tumor cells. The subtypes of glioma resemble the stages of glial stem cell differentiation. Tumor cells not readily differentiating makes them persistent, more mobile, harder to treat, and more aggressive.

Tumor cells fail to differentiate because their cellular metabolism is altered. A hallmark of gliomas is how they remodel metabolic pathways. Mutations in the membrane Na/K-ATPase or the enzymes of central metabolism skew the cellular reduction potential and Acetyl-CoA levels.

Metabolic remodeling has a cascading, inhibitory effect on cellular decision making. Remodeled metabolism in a tumor cells impairs its rate of differentiation, disables apoptosis, and promotes cellular motility. Cellular motility activates multiple synergistic mechanisms, which disable apoptosis. Ultimately, the process of cell migration produces digestion products of the brain extracellular matrix (ECM). These digestion product fragments promote stemness (stem cell like characteristics), promote aggressive growth, inhibit differentiation, and stimulate angiogenesis.

Conventional cancer treatments have repeatedly failed because they do not address the intracellular metabolic defects disabling cellular differentiation. Many different interventions, targeting metabolism, have shown some small improvement. However, effective therapies comprehensively and synergistically targeting cancer cell metabolism have not yet been developed to harness the potential of metabolic mechanisms to improve cancer treatment outcomes.

It would be desirable to have cancer treatment methods that address metabolic defects in cancer cells. In particular, it would be advantageous to develop cancer treatment methods that promote cancer cells differentiating.

Thus, there exists a need for methods for treating cancers that improve upon and advance the design of known cancer treatment methods. Examples of new and useful cancer treatment methods are discussed below.

### SUMMARY

The present disclosure is directed to cancer treatment methods. The cancer treatment methods include adjusting the in vivo concentration of a selected metabolite in a patient to levels effective to activate selected metabolic pathways that promote differentiation of cancer cells. In some examples, the methods for treating cancer include adjusting the in vivo concentration of one or more of vitamin-D, niacin, ascorbic acid, ascorbyl palmitate, and butyrate.

### BRIEF DESCRIPTION OF THE DRAWINGS

FIG. 1 is a flow diagram of a first example of a method for treating cancer.

FIG. 2 is a flow diagram of a metabolite concentration adjustment step of the method for treating cancer shown in FIG. 1.

FIG. 3 is a flow diagram of a metabolic pathways activation step of the method for treating cancer shown in FIG. 1.

### DETAILED DESCRIPTION

The disclosed methods for treating cancers will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various methods for treating cancers are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first” “second”, and “third” are used to distinguish or identify various members of a group, or the like, and are not intended to denote a serial, chronological, or numerical limitation.

“Coupled” means connected, either permanently or releasably, whether directly or indirectly through intervening components.

### Cancer Treatment Methods with Metabolite Adjustments

With reference to the figures, cancer treatment methods with metabolite adjustments will now be described. The methods discussed herein function to treat cancers by activating selected metabolic pathways. A wide range of cancer types may be treated with the methods discussed herein, inducing gliomas and glioblastomas.

The reader will appreciate from the figures and description below that the presently disclosed methods for treating cancers address many of the shortcomings of conventional methods for treating cancer. For example, the novel methods below address metabolic defects in cancer cells. More particularly, the novel methods below address metabolic defects in cancer cells that disable the cancer cells' tendency to differentiate. Causing cancer cells to more readily differentiate significantly improves cancer treatment outcomes.

### Cancer Treatment Method Embodiment One

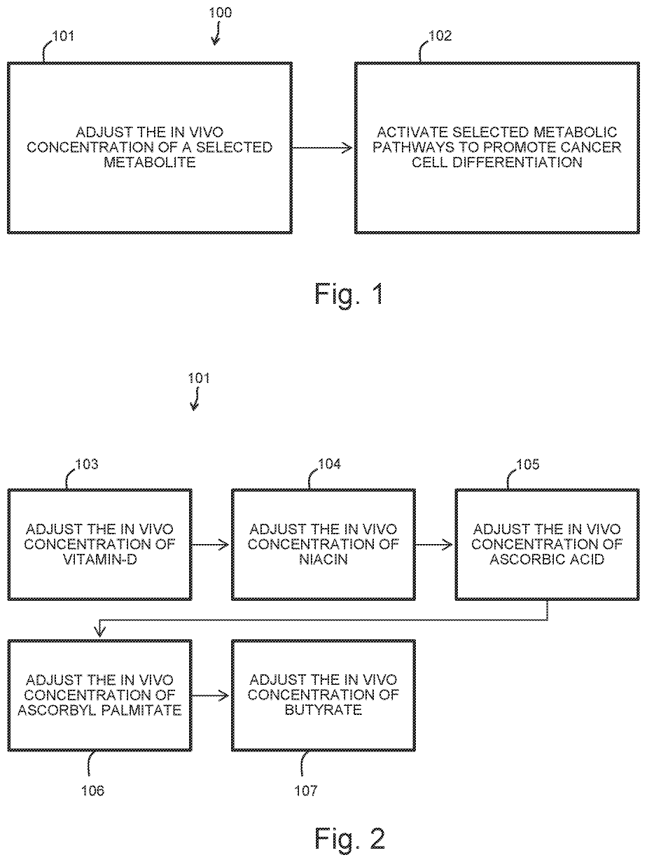

With reference to FIGS. 1-3, a first example of a method for treating cancer, method 100, will now be described. Method for treating cancer 100 includes adjusting the in vivo concentration of a selected metabolite at step 101 and activating selected metabolic pathways at step 102. In some examples, the method for treating cancer does not include the exact same steps shown in FIGS. 1-3 for method 100. In other examples, the method for treating cancer includes additional or alternative steps than shown in FIGS. 1-3.

### Adjusting In Vivo Metabolite Concentrations

Adjusting in vivo metabolite concentrations at step 101 functions to activate selected metabolic pathways at step 102. The selected metabolic pathways promote cancer cell differentiation and improve cancer treatment outcomes.

As can be seen in FIGS. 1 and 2, adjusting in vivo metabolite concentrations at step 101 includes adjusting the in vivo concentration of multiple metabolites; namely, vitamin-D at step 103, niacin at step 104, ascorbic acid at step 105, ascorbyl palmitate at step 106, and butyrate at step 107. However, the reader should understand that different variations of the method may include adjusting in vivo concentrations of fewer or additional metabolites. For example, some cancer treatment methods examples contemplated herein adjust a single metabolite, e.g., just vitamin-D, or a smaller subset of metabolites, e.g., just niacin and ascorbic acid. In certain examples, in vivo concentrations of additional or alternative metabolites beyond those expressly described herein are adjusted.

The in vivo concentrations of the selected metabolites may be adjusted to varying levels effective to activate one or more selected metabolic pathways. For example, the in vivo concentration of vitamin-D may be adjusted at step 103 to promote increased cAMP signaling at step 121. The in vivo concentration of niacin may be adjusted at step 104 to promote increased cAMP signaling at step 121 as well.

In the example shown in FIG. 2, the in vivo concentration of ascorbic acid is adjusted at step 105 to control the intracellular redox potential of cancer cells at step 122. Adjusting the in vivo concentration of ascorbic acid at step 105 also controls the nitrous oxide signaling pathway at step 123. Restoring the nitrous oxide signaling pathway at step 123 promotes apoptosis of cancer cells. Adjusting the in vivo concentration of ascorbic acid at step 105 also functions to restore a normal ratio of tetrahydrobiopterin to dihydrobiopterin at step 124.

At step 106, the in vivo concentration of ascorbyl palmitate is adjusted at step 106 to inhibit hyaluronidase at step 125. Adjusting the in vivo concentration of ascorbyl palmitate at step 106 also functions to inhibit glial stem cell migration at step 126. The step 106 adjustment of ascorbyl palmitate in vivo concentration further serves to beneficially reduce concentrations of low molecular weight hyaluronic acid at step 127. Adjusting the in vivo concentration of ascorbyl palmitate at step 106 beneficially reduces hyaluronic acid binding to CD44 glycoprotein.

Step 107 entails adjusting the in vivo concentration of butyrate to deplete bromo domain proteins at step 129. The adjustment of in vivo concentrations of butyrate at step 107 beneficially serves to upregulate toll-like receptor 4 at step 130. Adjusting the in vivo concentration of butyrate at step 107 also promotes differentiation of gliomas to glial cells at step 131.

### Activating Selected Metabolic Pathways

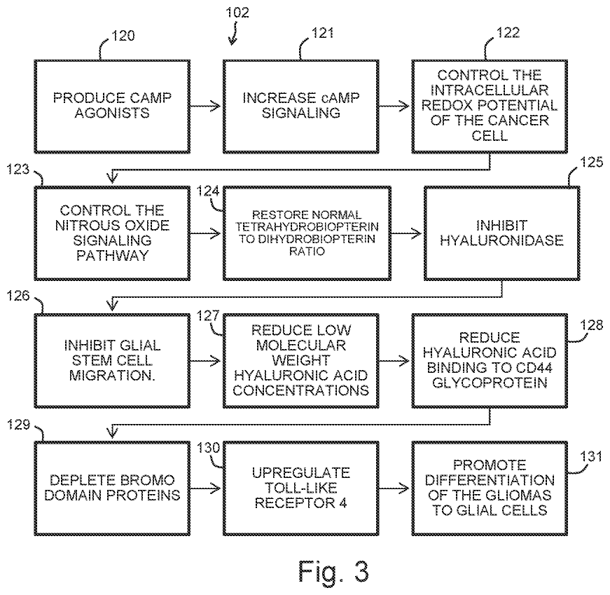

In the example shown in FIGS. 1 and 3, activating selected metabolic pathways at step 102 functions to promote therapeutically beneficial cancer cell differentiation. In some examples, the selected metabolic pathways correspond to cellular pathways used by a combination of a cAMP agonist, an mTOR inhibitor, and a bromo domain inhibitor. Cellular pathways corresponding to a three molecule combination of a cAMP agonist, are mTOR inhibitor, and a bromo domain inhibitor, induce differentiation of glioma to a glial cell. In certain examples, the metabolic pathways are selected to produce cAMP agonists.

Underlying biological mechanisms giving rise to the metabolic pathways promoting cancer cell differentiation are discussed below. References to published literature relevant to the mechanisms discussed below are also provided below and incorporated herein for further details of the mechanisms.

### Redox State

Redox potential controlled at step 122 can directly influence a cancer cell's decision to divide, differentiate, or commit apoptosis. Thiol redox controls multiple steps of apoptosis, including cathepsins and activation of caspases. Glutathione (GSH) controls a cancer cell's decision between apoptosis and survival and changing cellular glutathione levels sensitizes cancer cells to apoptotic stimuli. The intracellular redox potential can be measured and influenced using ascorbate at step 105.

### Nitrous Oxide System (NOS)

Changes to redox state and glutathione, in turn, affect the nitrous oxide (NO) signaling pathway controlled at step 123. NO synthases become uncoupled in cancer and produce peroxynitrate. Nitrous oxide synthases becoming uncoupled turns on the pentose phosphate pathway (PPP), GSH synthesis, and glycolysis. PPP, GSH synthesis, and glycolysis promotes stemness and nucleotide synthesis. Aberrant function of the NOS system impairs execution of apoptosis and instead promotes survival of cancer cells.

The NO system is complex in its regulation, but NO synthase is regulated by the intracellular redox state via glutathione and tetrahydrobiopterin. The intracellular redox state determines NOS uncoupling by the ratio of reduced tetrahydrobiopterin (BH4) to dihydrobiopterin (BH2) at step 124 in the NO synthase. Restoration of the normal ratio at step 124 reverses this uncoupling, and this ratio can be influenced using ascorbic acid at step 105. The enzyme controlling tetrahydrobiopterin and dihydrobiopterin interconverting is elevated in aggressive glioma.

Inducible nitric oxide synthase has complex, context dependent effects. In glioma, iNOS-derived NO is key to metabolic remodeling and cytokine production in the pro-inflammatory macrophage. Nitrous oxide is one of the key molecules responsible for macrophage killing of glioblastomas. Perivascular eNOS expression is elevated in glioblastomas and upregulates Notch signaling to promote stemness. Perivascular eNOS expression is correlated with cancer cell aggressiveness.

### cGMP

The eNOS system activates the NO/cGMP/PKG pathway. This pathway is essential for MG neuron migration and is a key driver of cell motility in glioma. The NO/cGMP/PKG signaling cascade promotes aggressive growth and stemness in glioma and other cancers and significantly influences neural stem cell survival.

cGMP is produced by guanylate cyclase. Guanylate cyclase is a soluble NO receptor that amplifies the NO cGMP signal. The function of cCMP is regulated by redox state at step 122.

cGMP is broken down by phosphodiesterases (PDEs) and cGMP levels can be manipulated by influencing phosphodiesterase expression. Degradation of cGMP by PDE5 increases survival in glioblastoma. The NO/cGMP/PKG pathway upregulates the mitochondrial ascorbate transporter in cancer and vitamin-C kills these cancer stem cells.

Glioma differentiating to a glial cell requires a cAMP agonist. The cAMP/protein-kinase-A pathway opposes NO/cGMP cell migration in neural precursors. The cAMP pathway promotes expression of cyclin dependent kinase inhibitors p21 and p27. cAMP synergizes with Tor inhibition to reduce Myc/stemness signaling.

In contrast to cGMP, increasing cAMP or inhibiting cAMP degradation promotes better outcomes.

cAMP can also be optimized by repairing redox coupling of the NOS. Intermittent fasting and ketosis increases cAMP levels globally. COQ10 reduces expression of PDE4 and S-adenosylmethionine (SAMe) is a PDE4B inhibitor.

Vitamin D is important in differentiation in many cancers. Vitamin D synergizes with cAMP signaling to promote differentiation of cancer cells. Calcitriol induces differentiation in glioblastoma.

Niacin also increases cAMP signaling and increases the activity of sirtuins. Sirtuins are important for promoting differentiation of cancer cells.

### Motility

Motility inhibits apoptosis by multiple synergistic mechanisms. Like metabolism, cellular motility disables cellular processes, such as apoptosis and differentiation, by multiple independent mechanisms. The multiple, independent mechanisms amplify and compound each other.

Motility, and the resultant cell polarity, activates the protective Akt mTOR pathway and allows stem cell transcription factors to express. Motility disables apoptosis by controlling the cellular localization CDKIs (Cyclin Dependent Kinase Inhibitors). Motility forces cytosolic localization of CDKIs p27 and p21.

Cytosolic localization of the p27 and p21 CDKIs inhibits caspases, allows cMYC to express, and permits cell cycles to progress. different CDKI, p57Kip2, translocates to the nucleus and disables the mitochondrial apoptotic pathway. Thus, migration inhibitors are synergistic with chemotherapy regimens, inhibiting migration of glioma cells at step 126 promotes differentiation and apoptosis of glioma cells.

### Hyalurnoic Acid

With reference to steps 125-128, hyaluronic acid affects migration, stemness signaling, and angiogenesis in cancer cells. Hyaluronic acid is a sensor of tissue damage and metabolism and makes up a large portion of the brain ECM. Hyaluronidases localize to the growth cones of migrating glial stem cells.

Cell migration produces fragments of low molecular weight hyaluronic acid (LMWHA). LMWHA inhibit cellular differentiation and promote angiogenesis.

Hyaluronic acid binds to CD44, which interacts with cysteine glutamate antiporter. Cysteine glutamate antiporter is implicated in metabolic stemness associated with aggressive, undifferentiated cancers.

Hyaluronan-CD44 interacts with stem cell factors Oct4, Sox2, and Nanog to promote stemness. In glioma, LMWHA oligosaccharides promote cleaving CD44. The cleaved CD44 promotes aggressive growth.

### Ascorbyl Palmitate

Inhibiting hyaluronidases at step 125 potently inhibits glial stem cell migration at step 126. Ascorbyl palmitate adjusted at step 106 is a hyaluronidase inhibitor effective in the low-micromolar range. Ascorbyl palmitate accumulates in the central nervous system and is approved by the FDA as a food additive.

Ascorbyl palmitate is an excellent drug candidate for glioblastoma because it targets a trait specific to glioblastoma and critical for the spread of glioblastoma. Ascorbyl palmitate potently inhibits glial precursor cell migration at step 126 in slice cultures and promotes glial cell differentiation at step 131. Ascorbyl palmitate is safe, cheap, specific, and has multiple synergistic effects on stemness signaling and glioblastoma pathogenesis. The beneficial effects on stemness signaling and glioblastoma pathogenesis include addressing migration dependent resistance to apoptosis, the HA CD44 stemness pathways, and HA dependent angiogenesis.

### TLR4 Signaling

Toll-like receptors (TLRs) regulate neural stem cell proliferation. LMWHA promotes differentiation in glioblastoma stem cells by stimulating toll-like receptor 4 (TLR4) to activate NFϰ-B. Downregulating TLR4 promotes stem cell self-renewal and correlates with aggressiveness. Butyrate treatment at step 107 upregulates TLR4 at step 130 to oppose cancer cell aggression and promote cancer cells differentiating.

Butyrate is a histone deacetylase inhibitor that promotes tissue differentiation and modulates stemness. Butyrate selectively depletes bromo domain proteins at step 129. Selectively depleting bromo domain proteins at step 129 beneficially targets transcription of Myc. Inhibiting bromo domain proteins at step 129 is required for glioma to differentiate to glial cells.

Butyrate levels correlate with therapeutic response in anti-CTLA4 and anti-PD-L1 immunotherapy in melanoma. The antitumor response relies on toll-like receptor 4 signaling.

Butyrate inhibits cultured glioma cells proliferating, induces cellular differentiation, and inhibits invasiveness. Butyrate levels respond to dietary changes at step 107. Further, the HDAC activity of butyrate can be increased by supplements promoting fat metabolism.

### REFERENCES INCORPORATED BY REFERENCE

References relevant to cancer treatment methods include the following references, the complete disclosures of which are incorporated herein by reference for all purposes:

Strowd, R. E. & Grossman, S. A. The Role of Glucose Modulation and Dietary Supplementation in Patients With Central Nervous System Tumors. Curr. Treat. Options Oncol. 16, 36 (2015).

Phillips, H. S. et al. Molecular subclasses of high-grade glioma predict prognosis, delineate a pattern of disease progression, and resemble stages in neurogenesis. Cancer Cell 9, 157-173 (2006).

Strickland, M. & Stoll, E. A. Metabolic Reprogramming in Glioma. Front. Cell Dev. Biol. 5, (2017).

Lefranc, F. & Kiss, R. The Sodium Pump α1 Subunit as a Potential Target to Combat Apoptosis-Resistant Glioblastomas. Neoplasia 10, 198-206 (2008).

Reitman, Z. J. & Yan, H. Isocitrate dehydrogenase 1 and 2 mutations in cancer: alterations at a crossroads of cellular metabolism. J. Natl. Cancer Inst. 102, 932-41 (2010).

Dang, L. et al. Cancer-associated IDH1 mutations produce 2-hydroxyglutarate. Nature 462, 739-44 (2009).

Molina, J. R., Hayashi, Y., Stephens, C. & Georgescu, M.-M. Invasive Glioblastoma Cells Acquire Stemness and Increased Akt Activation. Neoplasia 12, 453-IN5 (2010).

Oh, J., Kim, Y., Baek, D. & Ha, Y. Malignant gliomas can be converted to nonproliferating glial cells by treatment with a combination of small molecules. Oncol. Rep. 41, 361-368 (2019).

Menon, S. G. & Goswami, P. C. A redox cycle within the cell cycle: Ring in the old with the new. Oncogene 26, 1101-1109 (2007).

Smith, J., Ladi, E., Mayer-Proschel, M. & Noble, M. Redox state is a central modulator of the balance between self-renewal and differentiation in a dividing glial precursor cell. Proc. Natl. Acad. Sci. U. S. A. 97, 10032-7 (2000).

Aquilano, K., Baldelli, S. & Ciriolo, M. R. Glutathione: New roles in redox signalling for an old antioxidant. Front. Pharmacol. 5 August, 1-12 (2014).

Hattori, K., Naguro, I., Runchel, C. & Ichijo, H. The roles of ASK family proteins in stress responses and diseases. Cell Commun. Signal. 7, 1-10 (2009).

Circu, M. L. & Aw, T. Y. Glutathione and modulation of cell apoptosis. Biochim. Biophys. Acta-Mol. Cell Res. 1823, 1767-1777 (2012).

Hwang, S. Y. et al. Induction of glioma apoptosis by microglia-secreted molecules: The role of nitric oxide and cathepsin B. Biochim. Biophys. Acta-Mol. Cell Res. 1793, 1656-1668 (2009).

Hentze, H. et al. Glutathione dependence of caspase-8 activation at the death-inducing signaling complex. J. Biol. Chem. 277, 5588-5595 (2002).

De Nicola, M. & Ghibelli, L. Glutathione depletion in survival and apoptotic pathways. Front. Pharmacol. 5, 1-4 (2014).

Bohndiek, S. E. et al. Hyperpolarized [1-13C]-ascorbic and dehydroascorbic acid: Vitamin C as a probe for imaging redox status in vivo. J. Am. Chem. Soc. 133, 11795-11801 (2011).

Rabender, C. S. et al. The role of nitric oxide synthase uncoupling in tumor progression. Mol. Cancer Res. 13, 1034-1043 (2015).

Tran, A. N., Boyd, N. H., Walker, K. & Hjelmeland, A. B. NOS Expression and NO Function in Glioma and Implications for Patient Therapies. Antioxid. Redox Signal. 26, 986-999 (2016).

Bolaños, J. P., Delgado-Esteban, M., Herrero-Mendez, A., Fernandez-Fernandez, S. & Almeida, A. Regulation of glycolysis and pentose-phosphate pathway by nitric oxide: Impact on neuronal survival. Biochim. Biophys. Acta-Bioenerg. 1777, 789-793 (2008).

Bailey, J. D. et al. Nitric Oxide Modulates Metabolic Remodeling in Inflammatory Macrophages through TCA Cycle Regulation and Itaconate Accumulation. Cell Rep. 28, 218-230.e7 (2019).

Förstermann, U. & Sessa, C. Nitric oxide synthases: Regulation and function. Eur. Heart J. 33, 829-837 (2012).

Mintz, J. et al. Current advances of nitric oxide in cancer and anticancer therapeutics. Vaccines 9, 1-39 (2021).

Nikulina, M. A. et al. GLUTATHIONE DEPLETION INHIBITS IL-1β-STIMULATED NITRIC OXIDE PRODUCTION BY REDUCING INDUCIBLE NITRIC OXIDE SYNTHASE GENE EXPRESSION. Cytokine 12, 1391-1394 (2000).

d'Uscio, L. V., Milstien, S., Richardson, D., Smith, L. & Katusic, Z. S. Long-term vitamin C treatment increases vascular tetrahydrobiopterin levels and nitric oxide synthase activity. Circ. Res. 92, 88-95 (2003).

Tran, A. N. et al. Reactive species balance via GTP cyclohydrolase i regulates glioblastoma growth and tumor initiating cell maintenance. Neuro. Oncol. 20, 1055-1067 (2018).

Vannini, F., Kash, K. & Nath, N. Redox Biology The dual role of iNOS cancer $. 6, 334-343 (2015).

Viola, A., Munari, F., Sánchez-Rodríguez, R., Scolaro, T. & Castegna, A. The metabolic signature of macrophage responses. Front. Immunol. 10, 1-16 (2019).

Charles, N. et al. Perivascular Nitric Oxide Activates Notch Signaling and Promotes Stem-like Character in PDGF-Induced Glioma Cells. Cell Stem Cell 6, 141-152 (2010).

Zhu, H. et al. Restoring soluble guanylyl cyclase expression and function blocks the aggressive course of glioma. Mol. Pharmacol. 80, 1076-1084 (2011).

Codrici, E., Enciu, A. M., Popescu, I. D., Mihai, S. & Tanase, C. Glioma Stem Cells and Their Microenvironments: Providers of Challenging Therapeutic Targets. Stem. Cells Int. 2016, (2016).

Bian, K. et al. NO-cGMP signalling and cancer therapy. BMC Pharmacol. Toxicol. 14, P9 (2013).

Wang, R. et al. iNOS promotes CD24+CD133+ liver cancer stem cell phenotype through a TACE/ADAM17-dependent Notch signaling pathway. Proc. Natl. Acad. Sci. 115, E10127-E10136 (2018).

Fiscus, R. R. Involvement of cyclic GMP and protein kinase G in the regulation of apoptosis an survival in neural cells. NeuroSignals 11, 175-190 (2002).

Beuve, A. Thiol-Based Redox Modulation of Soluble Guanylyl Cyclase, the Nitric Oxide Receptor. Antioxidants acid Redox Signaling 26, 137-149 (2017).

Stuehr, D. J., Misra, S., Dai, & Ghosh, A. Maturation, inactivation, and recovery mechanisms of soluble guanylyl cyclase. J. Biol. Chem. 29, 100336 (2021).

Sun, J. et al. Advances in Cyclic Nucleotide Phosphodiesterase-Targeted PET Imaging and Drug Discovery. J. Med. Chem. 64, 7083-7109 (2021).

Piazza, G. A. et al. PDE5 and PDE10 inhibition activates cGMP/PKG signaling to block Wnt/β-catenin transcription, cancer cell growth, and tumor immunity. Drug Discov. Today 25, 1521-1527 (2020).

Cesarini, V. et al. Type 5 phosphodiesterase regulates glioblastoma multiforme aggressiveness and clinical outcome. Oncotarget 8, 13223-13239 (2017).

Portugal, C. C. et al. Nitric oxide modulates sodium vitamin C transporter 2 (SVCT-2) protein expression via protein kinase C (PKG) and nuclear factor-ϰB (NF-ϰB). J. Biol. Chem. 287, 3860-3872 (2012).

Roa, F. J. et al. Data on SVCT2 transporter expression and localization in cancer cell lines and tissues. Data Br. 25, 1-6 (2019).

Peña, E. et al. Increased expression of mitochondria sodium-coupled ascorbic acid transporter-2 (mitSVCT2) as a central feature in breast cancer. Free Radic. Biol. Med. 135, 283-292 (2019).

Haase, A. & Bicker, G. Nitric oxide and cyclic nucleotides are regulators of neuronal migration in an insect embryo. Development 130, 3977-3987 (2003).

Sassone-Corsi, P. The Cyclic AMP pathway. Cold Spring Harb. Perspect. Biol, 4, 3-5 (2012).

Chen, T. C. et al. The type IV phosphodiesterase inhibitor rolipram induces expression of the cell cycle inhibitors p21(Cip1) and p27(Kip1), resulting in growth inhibition, increased differentiation, and subsequent apoptosis of malignant A-172 glioma cells. Cancer Biol. Ther. 1, 268-76 (2002).

Leslie, S. N. & Nairn, A. C. cAMP regulation of protein phosphatases PP1 and PP2A in brain. Biochim. Biophys. Acta-Mol. Cell Res. 1866, 64-73 (2019).

Allen-Petersen, B. L. et al. Activation of PP2A and inhibition of mTOR synergistically reduce MYC signaling and decrease tumor growth in pancreatic ductal adenocarcinoma. Cancer Res. 79, 209-219 (2019).

Xu, Z. et al. Coenzyme Q10 improves lipid metabolism and ameliorates obesity by regulating CaMKIIMediated PDE4 inhibition. Sci. Rep. 7, 1-12 (2017).

Patra, C., Foster, K., Corley, J. E., Dimri, M. & Brady, M. F. Biochemistry, cAMP. in (2021).

Rubin, J. E., Chenoweth, D. E. & Catherwood, B. D. 1,25-Dihydroxyvitamin D3 and adenosine 3′,5′-monophosphate synergistically promote differentiation of a monocytic cell line. Endocrinology 118, 2540-2545 (1986).

Gocek, E. & Studzinski, G. P. Vitamin D and differentiation in cancer Signaling differentiation E. Gocek and G. P. Studzinski. Crit. Rev. Clin. Lab. Sci. 190-209 (2009).

Gerstmeier, J. et al. Calcitriol promotes differentiation of glioma stem-like cells and increases their susceptibility to temozolomide. Cancers 13, (2021).

Kotliarova, S. et al. Glycogen synthase kinase-3 inhibition induces glioma cell death through c-MYC, nuclear factor-ϰB and glucose regulation. Cancer Res. 68, 6643-6651 (2008).

Besson, A., Dowdy, S. F. & Roberts, J. M. CDK inhibitors: cell cycle regulators and beyond. Dev. Cell 14, 159-69 (2008).

Vlachos P., Nyman, U., Hajji, N. & Joseph, B. The cell cycle inhibitor p57(Kip2) promotes cell death via the mitochondrial apoptotic pathway. Cell Death Differ. 14, 1497-507 (2007).

Lefranc, F., Brotchi, J. & Kiss, R. Possible future issues in the treatment of glioblastomas: special emphasis on cell migration and the resistance of migrating glioblastomit cells to apoptosis. J. Clin. Oncol. 23, 2411-22 (2005).

Garantziotis, S. & Savani, R. C. Hyaluronan biology: A complex balancing act of structure function, location and context. Matrix Biol. 78-79, 1-10 (2019).

Sloane, J. a. et al. Hyaluronan blocks oligodendrocyte progenitor maturation and remyelination through TLR2. Proc. Natl. Acad. Sci. 107, 11555-11560 (2010).

Back, S, a et al. Hyaluronan accumulates in demyelinated lesions and inhibits oligodendrocyte progenitor maturation. Nat. Med, 11, 966-72 (2005).

Kaessler, A., Nourrisson, M.-R., Duflos, M. & Jose, J. Indole carboxamides inhibit bovine testes hyaluronidase at pH 7.0 and indole acetamides activate the enzyme at pH 3.5 by different mechanisms. J. Enzyme Inhib. Med. Chem. 23, 719-27 (2008).

Slevin, M. et al. Hyaluronan-mediated angiogenesis in vascular disease: Uncovering RHAMM and CD44 receptor signaling pathways. 26, 58-68 (2007).

Akiyama, Y. et al. Hyaluronate receptors mediating glioma cell migration and proliferation. J. Neurooncol. 53, 115-27 (2001).

Liu, D. et al. Expression of hyaluronidase by tumor cells induces angiogenesis in vivo. Proc. Natl. Acad. Sci. U. S. A. 93, 7832-7 (1996).

Hasegawa, M. et al. Functional interactions of the cystine/glutamate antiporter, CD44V and MUC1-C oncoprotein in triple-negative breast cancer cells. Oncotarget 7, 1175-11769 (2016).

Bourguignon, L. Y. W., Wong, G., Earle, C. & Chen, L. Hyaluronan-CD44v3 interaction with Oct4-Sox2-Nanog promotes miR-302 expression leading to self-renewal, clonal formation, and cisplatin resistance in cancer stem cells from head and neck squamous cell carcinoma. J. Biol. Chem. 287, 32800-32824 (2012).

Bourguignon L. Y. W., Wong, G., Earle, C. & Chen, L. Hyaluronan-CD44v3 interaction with Oct4-Sox2-Nanog promotes miR-302 expression leading to self-renewal, clonal formation, and cisplatin resistance in cancer stem cells from head and neck squamous cell carcinoma. Biol. Chem. 287, 32800-32824 (2012).

Louderbough, J. M. V. & Schroeder, J. A. Understanding the dual nature of CD44 in breast cancer progression. Mol. Cancer Res. 9, 1573-1586 (2011).

Pietras A. et al. Osteopontin-CD44 signaling in the glioma perivascular niche enhances cancer stem cell phenotypes and promotes aggressive tumor growth. Cell Stem Cell 14, 357-369 (2014).

Ölgen, S., Kaeßler, A., Nebio\grave{g}lu, D. & Jose, J. Research Letter: New Potent Indole Derivatives as Hyaluronidase Inhibitors. Chem. Biol. Drug Des. 70, 547-551 (2007).

Naidu, a K., Wiranowska, M., Kori, S. H., Prockop, L. D. & Kulkarni, a P. Inhibition of human glioma cell proliferation and glutathione S-trase by ascorbyl esters and interferon. Anticancer Res 13, 1469-1475 (1993).

Preston, M. et al. Digestion products of the PH20 hyaluronidase inhibit remyelination. Ann. Neurol. 73, 266-280 (2013).

Yaddanapudi, K., Miranda, J. De, Hornig, M. & Lipkin, W. I. Toll-like Receptor 3 Regulates Neural Stem Cell Proliferation by Modulating the Sonic Hedgehog Pathway. 6, (2011).

Ferrandez, E., Gutierrez, O., Segundo, D. S. & Fernandez-Luna, J. L. NFϰB activation in differentiating glioblastoma stem-like cells is promoted by hyaluronic acid signaling through TLR4. Sci. Rep. 8, 6341 (2018).

Alvarado, A. G. et al. Glioblastoma Cancer Stem Cells Evade Innate Immune Suppression of Self-Renewal through Reduced TLR4 Expression. Cell Stem Cell 20, 450-461.e4 (2017).

Xiao, T. et al. Butyrate upregulates the TLR4 expression and the phosphorylation of MAPKs and NK-ϰB in colon cancer cell in vitro. Oncol. Lett. 16, 4439-4447 (2018).

Nagpal, R. et al. Human-origin probiotic cocktail increases short-chain fatty acid production via modulation of mice and human gut microbiome. Sci. Rep. 8, 12649 (2018).

Wang, Q. et al. Ketogenesis contributes to intestinal cell differentiation. Cell Death Differ. 24, 458-468 (2017).

Mackmull, M. T. et al. Histone deacetylase inhibitors (HDACi) cause the selective depletion of bromodomain containing proteins (BCPs). Mol. Cell. Proteomics 14, 1350-1360 (2015).

Delmore, J. E. et al. BET bromodomain inhibition as a therapeutic strategy to target c-Myc. Cell 146, 904-917 (2011).

Oh, J., Kim, Y., Baek, D. & Ha, Y. Malignant gliomas can be converted to non-proliferating glial cells by treatment with a combination of small molecules. Oncol Rep 41, 361-368 (2019).

Chaput, N. et al. Baseline gut microbiota predicts clinical response and colitis in metastatic melanoma patients treated with ipilimumab. Ann. Oncol. 28, 1368-1319 (2017).

Gopalakrishrian, V. et al. Gut microbiome modulates response to anti-PD-1 immunotherapy in melanoma patients. Science (80-.). 359, 97-103 (2018).

Pfirschke, C. et al. Immunogenic Chemotherapy Sensitizes Tumors to Checkpoint Blockade Therapy. Immunity 44, 343-354 (2016).

Engelhard, H. H., Duncan, H. A., Kim, S., Criswell, P. S. & Van Eldik, L. Therapeutic Effects of Sodium Butyrate on Glioma Cells in Vitro and in the Rat C6 Glioma Model. Neurosurgery 48, 616-625 (2001).

Nakagawa, H., Sasagawa, S. & Itoh, K. Sodium butyrate induces senescence and inhibits the invasiveness of glioblastoma cells. Oncol. Lett. 15, 1495-1502 (2018).

Castro, M. a., Beltrán, F. a., Brauchi, S. & Concha, I. I. A metabolic switch in brain: glucose and lactate metabolism modulation by ascorbic acid. J. Neurochem. 110, 423-140 (2009).

d'Uscio, L. V., Milstien S., Richardson, D., Smith, L. & Katusic, Z. S. Long-term vitamin C treatment increases vascular tetrahydrobiopterin levels and nitric oxide synthase activity. Circ. Res. 92, 88-95 (2003).

El Banna, N. et al. Redox modifications of cysteine-containing proteins, cell cycle arrest and translation inhibition: Involvement in vitamin C-induced breast cancer cell death. Redox Biol. 26, 101290 (2019).

Nauman, G., Gray, J. C., Parkinson, R., Levine, M. & Paller, C. J. Systematic review of intravenous ascorbate in cancer clinical trials. Antioxidants 7, 1-22 (2018).

Stephenson, C. M., Levin, R. D., Spector, T. & Lis, C. G. Phase i clinical trial to evaluate the safety, tolerability, and pharmacokinetics of high-dose intravenous ascorbic acid in patients with advanced cancer. Cancer Chemother. Pharmacol. 72, 139-146 (2013).

Stuhr, L. E. B. et al. Hyperoxia retards growth and induces apoptosis, changes in vascular density and gene expression in transplanted gliomas in nude rats. J. Neurooncol. 85, 191-202 (2007).

Bennett, M. H., Feldmeier, J., Smee, R. & Milross, C. Hyperbaric oxygenation for tumour sensitisation to radiotherapy. Diving Hyperb. Med. 48, 116-117 (2018).

Stępień, K., Ostrowski, R. P. & Matyja, E. Hyperbaric oxygen as an adjunctive therapy in treatment of malignancies, including brain tumours. Med. Oncol. 33, 1-9 (2016).

Ogawa, K. et al. Phase II trial of radiotherapy after hyperbaric oxygenation with chemotherapy for high-grade gliomas. Br. J. Cancer 95, 862-868 (2006).

Stacpoole, P. W. Therapeutic Targeting of the Pyruvate Dehydrogenase Complex/Pyruvate Dehydrogenase Kinase (PDC/PDK) Axis in Cancer. J. Natl. Cancer Inst. 109, 1-14 (2017).

Yue, L. et al, α-Lipoic Acid Targeting PDK1/NRF2 Axis Contributes to the Apoptosis Effect of Lung Cancer Cells. Oxid. Med. Cell. Longev. 2021, (2021).

Solmonson, A. & DeBerardinis, R. J. Lipoic acid metabolism and mitochondrial redox regulation. J. Biol. Chem. 293, 7522-7530 (2018).

Han, A., Bennett, N., Ahmed, B., Whelan, J. & Donohoe, D. R. Butyrate decreases its own oxidation in colorectal cancer cells through inhibition of histone deacetylases. Oncotarget 9, 27280-27292 (2018).

Han, A. et al. Cellular Metabolism and Dose Reveal Carnitine-Dependent and -Independent Mechanisms of Butyrate Oxidation in Colorectal Cancer Cells. J. Cell. Physiol. 231, 1804-1813 (2016).

Li, J. et al. Nicotinic acid inhibits glioma invasion facilitating Snail1 degradation. Sci. Rep. 7, 1-12 (2017).

Sarkar, S. et al. Control of brain tumor growth by reactivating myeloid cells with niacin. Sci. Transl. Med. 12, (2020).

Cataldi, S. et al. Effect of 1α,25(OH)2 vitamin D3 in mutant P53 glioblastoma cells: Involvement of neutral sphingomyelinase1. Cancers 12, 1-15 (2020).

Norlin, M. Effects of vitamin D in the nervous system: Special focus on interaction with teroid hormone signaling and a possible role in the treatment of brain cancer. J. Neuroendocrinol. 32, e12799 (2020).

Yang, Y. S., Yang, S. Li, D. & Li, W. Vitamin D affects the Warburg effect and stemness maintenance of non-small-cell lung cancer cells by regulating PI3K/AKT/mTOR signaling pathway. Curr. Cancer Drug Targets (2021). doi:10.2174/1568009621666210729100300

Shan, N. L. et al. Vitamin D compounds inhibit cancer stem-like cells and induce differentiation in triple negative breast cancer. J. Steroid Biochem. Mol. Biol. 173, 122-129 (2017).

Penet, M, F. et al. Effect of Pantethine on Ovarian Tumor Progression and Choline Metabolism. Front. Oncol. 6, 1-9 (2016).

Woolf, E. C. & Scheck, A. C. The ketogenic diet for the treatment of malignant glioma. J. Lipid Res. 56, 5-10 (2015).

Varshneya, K., Carico, C., Ortega, A. & Patil, C. G. The Efficacy of Ketogenic Diet and Associated Hypoglycemia as an Adjuvant Therapy for High-Grade Gliomas: A Review of the Literature. Cureus 7, 1-12 (2015).

Rabender, C. S. et al. Sepiapterin enhances tumor radio- and chemosensitivities by promoting vascular normalization. J. Pharmacol. Exp. Ther. 365, 536-543 (2018).

Zgheib, R. et al. Folate can promote the methionine-dependent reprogramming of glioblastoma cells towards pluripotency. Cell Death Dis. 10, (2019).

Ghannad-Zadeh, K. & Das, S. One-carbon metabolism associated vulnerabilities in glioblastoma: A review. Cancers (Basel). 13, 1-14 (2021).

Gao, X. et al. Dietary methionine influences therapy in mouse cancer models and alters human metabolism. Nature 572, 397 101 (2019).

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A method for treating cancer comprising adjusting the in vivo concentration of a selected metabolite in a patient to levels effective to activate selected metabolic pathways that promote differentiation of cancer cells.

2. The method for treating cancer of claim 1, wherein:
the cancer includes a glioma, and
the selected metabolic pathway activated by adjusting the in vivo concentration at the selected metabolite promotes the glioma differentiating to a glial cell.

3. The method for treating cancer of claim 2, wherein the selected metabolic pathways correspond to cellular pathways used by a combination of a cAMP agonist, mTOR inhibitor, and a bromo domain inhibitor.

4. The method for treating cancer of claim 2, wherein the selected metabolic pathways produce cAMP agonists.

5. The method for treating cancer of claim 2, wherein:
the selected metabolite is vitamin-D; and
the in vivo concentration of vitamin D is increased to promote increased cAMP signaling.

6. The method for treating cancer of claim 2, wherein
the selected metabolite is niacin;
the in vivo concentration of niacin is increased to a level effective to increase cAMP signaling.

7. The method for treating cancer of claim 1, wherein the selected metabolite is ascorbic acid.

8. The method for treating cancer of claim 7, wherein the selected metabolic pathways control the intracellular redox potential of the cancer cell.

9. The method for treating cancer of claim 7, wherein the selected metabolic pathways control the nitrous oxide signaling pathway.

10. The method for treating cancer of claim 9, wherein the nitrous oxide signaling pathway is restored to promote apoptosis of the cancer cells.

11. The method for treating cancer of claim 7, wherein the in vivo concentration of ascorbic acid is adjusted to a level effective to restore a normal ratio of tetrahydrobiopterin to dihydrobiopterin.

12. The method for treating cancer of claim 1, wherein:
the selected metabolite is ascorbyl palmitate;
the in vivo concentration of ascorbyl palmitate is adjusted to a level effective to inhibit hyaluronidase.

13. The method for treating cancer of claim 12, wherein the in vivo concentration of ascorbyl palmitate is adjusted to a level effective to inhibit glial stem cell migration.

14. The method for treating cancer of claim 13, wherein the cancer cells include glioblastomas.

15. The method for treating cancer of claim 12, wherein the in vivo concentration of ascorbyl palmitate is adjusted to a level effective to reduce concentrations of low molecular weight hyaluronic acid.

16. The method for treating cancer of claim 12 wherein the in vivo concentration of ascorbyl palmitate is adjusted to a level effective to reduce hyaluronic acid binding to CD44 glycoprotein.

17. The method for treating cancer of claim 1, wherein:
the selected metabolite is butyrate;
the in vivo concentration of butyrate is adjusted to a level effective to deplete bromo domain proteins.

18. The method for treating cancer of claim 17, wherein:
the cancer cells include gliomas; and
the in vivo concentration of butyrate is adjusted to a level effective to promote differentiation of the gliomas to glial cells.

19. The method for treating cancer of claim 17, wherein the in vivo concentration of butyrate is adjusted to a level effective to upregulate toll-like receptor 4.

20. A method for treating cancer comprising adjusting the in vivo concentration of one or more of vitamin-D, niacin, ascorbic acid, ascorbyl palmitate, and butyrate in a patient to levels effective to activate selected metabolic pathways that promote differentiation of cancer cells.
